(19) United States
(12) Patent Application Publication
(10) Pub. No.: US 2021/0401905 A1
(43) Pub. Date: Dec. 30, 2021

(54) COMBINATION OF LACTOBACILLI FOR THE RELIEF OF IRRITABLE BOWEL SYNDROME AND FOR THE RELIEF OF OTHER GASTROINTESTINAL DISORDERS

(71) Applicant: Kerry Luxembourg S.à.r.l., Luxembourg (LU)

(72) Inventor: Serge CARRIÈRE, Verdun (CA)

(73) Assignee: Kerry Luxembourg S.à.r.l, Luxembourg (LU)

(21) Appl. No.: 16/978,575

(22) PCT Filed: Feb. 28, 2019
(86) PCT No.: PCT/IB2019/051627

§ 371 Date: Sep. 4, 2020

Related U.S. Application Data
(60) Provisional application No. 62/638,521, filed on Mar. 5, 2018.

(51) Int. Cl. A61K 35/747; A23L 33/135; A61P 1/00; A61K 9/48; A23L 33/185; A23L 33/19

(52) U.S. Cl. CPC A61K 35/747; A23L 33/135; A23L 33/19; A61K 9/48; A23L 33/185; A61P 1/00

(57) ABSTRACT

Described herein are compositions and methods relating to the use of a combination of live lactobacilli bacteria, particularly live Lactobacillus acidophilus, live Lactobacillus casei, and live Lactobacillus rhamnosus, for the relief of undesirable gastromtestmal health problems such as irritable bowel syndrome (IBS), abdominal pain, abdominal discomfort, bloating, liquid stools and constipation. This combination of live lactobacilli bacteria can also be used for improving quality of life of a subject suffering from IBS, for the relief of IBS, and/or for the prevention and/or treatment of IBS.

### CROSS-REFERENCE TO RELATED APPLICATIONS

This application is a national phase application under 35 U.S.C. § 371 of PCT International Application No. PCT/IB2019/051627, filed Feb. 28, 2019, which claims the benefit of U.S. Provisional Application No. 62/638,521, filed Mar. 5, 2018, each of which are herein incorporated by reference.

### FIELD OF THE INVENTION

The invention relates to the field of gastrointestinal disorders, and more particularly to the use of a combination of live lactobacilli bacteria for the relief of undesirable gastrointestinal health problems such as irritable bowel syndrome (IBS), abdominal pain, abdominal discomfort, bloating, liquid stools and constipation.

### BACKGROUND OF THE INVENTION

Irritable bowel syndrome (IBS) is a chronic, relapsing gastrointestinal disorder that affects 5-20% of the American population. A number of risk factors for IBS have been identified, including female sex, psychological problems, stress, food intolerance, and bacterial overgrowth of the small intestine (Aagaard et al., 2013). The cardinal symptoms of IBS include abdominal pain, bloating, and changes in bowel habits (Aagaard et al., 2013). The pathophysiology is defined and no intestinal structural abnormalities accompany the syndrome. The quality of life of individuals with IBS is severely impaired, with major impacts on the health care system and visits to primary care physicians and gastroenterologists (Coffin et al., 2004). In fact, IBS is the most frequent diagnosis in gastroenterology practices and one of the most frequent diagnoses in primary care practices (Peery et al., 2012). Based on specific symptomatology, patients with IBS can be sub-classified into three major groups: constipation-predominant (IBS-C), diarrhea-predominant (IBS-D), and mixed bowel patterns (IBS-M), each with an approximately equal distribution. These IBS symptoms are troubling to patients, result in lower quality of life, and interfere with social interactions (Coffin et al., 2004).

The ultimate treatment goal for IBS is to provide relief for the multiple symptoms of this condition by using a single, well-tolerated agent. Drug therapies may alleviate some of the symptoms linked with this condition, but none are curative. Therefore, the prospect of long-term treatment efficacy is limited given the current treatment options. There is a clear need for IBS relief procedures that are safe, efficacious, and cost effective (Foxx-Orenstein, 2006).

Probiotics are live micro-organisms that provide health benefits for the host when administered in adequate dosages. In recent years, probiotics have been commonly used to alleviate symptoms in a variety of gastrointestinal disorders. Since dysbiosis may be part of the multifactorial etiology of IBS, a variety of probiotics have been tested in clinical trials to determine their efficiency and the results have been included in several meta-analyses and review articles (Ford et al., 2014b; Hoveyda et al., 2009; McFarland and Dublin, 2008; Ortiz-Lucas et al., 2013; Whelan and Myers, 2010; Yoon et al., 2015). No firm conclusions could be drawn as to the efficacy of strain-specific probiotics for alleviating the symptoms of IBS. Strong placebo effects, psychological factors, and gender effects make the interpretation of study findings difficult (Ford and Moayyedi, 2010; Lyra et al., 2016; Moayyedi et al., 2010).

Probiotic products containing lactobacilli and bifidobacteria, have already been tested to improve IBS (Niv et al., 2005; O'Mahony et al., 2005). Positive results have been noted with some Lactobacillus strains, for example by Ducrotté et al, who reported resolution of all IBS-dominant symptoms, including abdominal pain, in 214 patients treated for 4 weeks with L. plantarum 299V (Ducrotté et al., 2012). Halpern et al. noted a significant reduction in an IBS symptoms index with a capsule containing 5×10⁹ heat-killed L. acidophilus (Halpern et al., 1996). Other Lactobacillus strains such as L. salivarius UCC4331 did not show any therapeutic gain over placebo in 75 patients (O'Mahony et al., 2005), nor did L. reuteri ATCC55730 (Niv et al., 2005), suggesting that some strains of Lactobacillus may be more effective than others in this indication.

Although products comprising Lactobacillus acidophilus, Lactobacillus casei and/or Lactobacillus rhamnosus strains have been tested during clinical research in the past (Beausoleil et al., 2007; Gao et al., 2010; Maziade et al., 2015; Sampalis et al., 2010), these clinical trials have never shown or suggested effectiveness of lactobacilli in the relief of irritable bowel syndrome (IBS), abdominal pain, bloating, and/or constipation.

Accordingly, there is a need for a combination of bacterial strains that are effective in relief of gastrointestinal disorders such as irritable bowel syndrome (IBS), abdominal pain, bloating, and/or constipation.

The present invention addresses these needs and other needs as it will be apparent from review of the disclosure and description of the features of the invention hereinafter.

### BRIEF SUMMARY OF THE INVENTION

The invention relates to the use of a combination of live lactobacilli bacteria for the relief of undesirable gastrointestinal health problems such as irritable bowel syndrome (IBS), abdominal pain, abdominal discomfort, bloating, liquid stools and constipation.

According to one particular aspect, the invention relates to a method for the relief of a gastrointestinal disorder in a subject in need thereof, comprising administering to said subject a combination of live Lactobacillus acidophilus, live Lactobacillus casei, and live Lactobacillus rhamnosus, wherein said gastrointestinal disorder is selected from the group consisting of irritable bowel syndrome (IBS), abdominal pain, bloating and constipation.

According to another particular aspect, the invention relates to a method for improving quality of life of a subject suffering from irritable bowel syndrome (IBS), comprising administering to said subject a combination of live Lactobacillus acidophilus, live Lactobacillus casei, and live Lactobacillus rhamnosus, wherein said administration provides to said subject at least one benefit selected from the group consisting of: satisfaction with bowel habit, minimal interference of IBS with normal activity, improved body image, reduction of food avoidance, increased social reaction, reduced sexual dysfunction, and improved relationships.

According to another particular aspect, the invention relates to a method for the relief of irritable bowel syndrome (IBS) in a human subject in need thereof, comprising administering to said subject a combination of live Lactobacillus acidophilus CL1285®, live Lactobacillus casei LBC80R® and live Lactobacillus rhamnosus CLR2®.

According to another particular aspect, the invention relates to an alternative method to drug therapy for the prevention and/or treatment of irritable bowel syndrome (IBS), comprising: (i) identifying a human subject in need of drug therapy for the prevention and/or treatment of IBS; and (ii) administering to said human subject a nutritionally acceptable composition comprising a combination of live micro-organisms comprising live Lactobacillus acidophilus, live Lactobacillus casei, and live Lactobacillus rhamnosus in addition or in replacement of said drug therapy.

According to another particular aspect, the invention relates to a composition for the relief of a gastrointestinal disorder in a subject in need thereof, the composition comprising a combination of live Lactobacillus acidophilus CL1285®, live Lactobacillus casei LBC80R®, live Lactobacillus rhamnosus CLR2®, wherein said gastrointestinal disorder is selected from the group consisting of irritable bowel syndrome (IBS), abdominal pain, days of pain, distention, stool consistency, and stool frequency.

According to another particular aspect, the invention relates to a composition for the relief of irritable bowel syndrome (IBS) in a subject in need thereof, the composition comprising a combination of live Lactobacillus acidophilus CL1285®, live Lactobacillus casei LBC80R® and live Lactobacillus rhamnosus CLR2®.

According to another particular aspect, the invention relates to the use of a combination of live Lactobacillus acidophilus, live Lactobacillus casei, and live Lactobacillus rhamnosus, for the relief of a gastrointestinal disorder in a subject in need thereof, wherein said gastrointestinal disorder is selected from the group consisting of irritable bowel syndrome (IBS), abdominal pain, bloating and constipation.

According to another particular aspect, the invention relates to the use of a combination of live Lactobacillus acidophilus CL1285®, live Lactobacillus casei LBC80R® and live Lactobacillus rhamnosus CLR2®, for the prevention, the treatment and/or the relief of irritable bowel syndrome (IBS) in a subject in need thereof.

### DETAILED DESCRIPTION OF PREFERRED EMBODIMENTS

The invention relates to the prevention, treatment and/or relief of gastrointestinal disorders in subjects.

As used herein, the term “gastrointestinal disorder” refers to gastrointestinal disorders which are characterized by symptoms such as abdominal pain, bloating, and constipation (e.g. stool consistency and frequency). The term encompasses, but is not limited to, irritable bowel syndrome (IBS), which includes constipation-predominant IBS (IBS-C), diarrhea-predominant IBS (IBS-D), and mixed bowel patterns IBS (IBS-M).

As used herein, the term “relief of a gastrointestinal disorder” or “relief of irritable bowel syndrome” or “relief of IBS” encompasses health benefits including, but not limited to, stabilizing, curing, healing, alleviating, relieving, altering, remedying, less worsening, ameliorating, improving, or affecting the disease or condition, the symptom of the disease or condition, or the risk of (or susceptibility to) the disease or condition. The term encompasses at least the relief of undesirable health problems including, but not limited to abdominal pain, extended duration of abdominal pain (consecutive or not), bloating, problems of stool consistency and/or frequency (e.g. constipation), reduced quality of life (QOL) associated with one or more of the above, and inadequate relief of such health issues when treated with medications or others.

As used herein, the term “subject” includes living organisms in which gastrointestinal disorder(s) may occur. The term “subject” includes animals (e.g., mammals (e.g., cats, dogs, horses, pigs, cows, goats, sheep, rodents (e.g., mice or rats), rabbits, squirrels, bears, primates (e.g., chimpanzees, monkeys, gorillas, and humans)), as well as avian (e.g. chickens, ducks, Peking ducks, geese), and transgenic species thereof. Preferably, the subject is a human or a non-human primate (e.g., chimpanzee, monkey, macaque, gorilla). More preferably, the subject is a human. Even more preferably the subject is a human in need of treatment and having, or likely to have, one of more undesirable health problems and/or symptoms such as abdominal pain, bloating, and constipation.

According to one particular aspect, the invention provides for the use of a combination of live Lactobacilli for the prevention, the treatment and/or the relief gastrointestinal disorders, particularly IBS. According to one particular embodiment, the combination of live Lactobacilli comprises live Lactobacillus acidophilus, live Lactobacillus casei and live Lactobacillus rhamnosus.

In embodiments, the combination comprises about 1-10% L. acidophilus, 70-90% L. casei and about 5-20% L. rhamnosus of the colony forming units (CFU) of the combination.

In one particular embodiment, the Lactobacillus acidophilus is Lactobacillus acidophilus CL1285® deposited at the National Collection of Microorganisms Cultures (CNCM) in Paris, deposit No. CNCM 1-4099, the Lactobacillus casei is Lactobacillus casei LBC80R® deposited at the CNCM as deposit No. CNCM 1-3989, and the Lactobacillus rhamnosus is Lactobacillus rhamnosus CLR2® deposited at the CNCM as deposit No. CNCM 1-3990.

In embodiments, the invention comprises administering simultaneously at least 10 billion, or at least 20 billion, or at least 30 billion, or at least 40 billion, or at least 50 billion, or at least 75 billion, or at least 100 billion, or at least 150 billion, or at least 200 billion of said combination of Lactobacilli.

In embodiments, the invention comprises administering the combination of live Lactobacilli once per day, twice per day, three times per day or more.

In embodiments, the combination of live Lactobacilli is administered as a nutritionally acceptable composition. As used herein the term “nutritionally acceptable composition” refers to a substance, e.g. a food substance, which will provide once ingested, nutritional support and nutrients such as carbohydrates, fats, proteins, vitamins, and/or minerals etc. Once ingested, in addition to provide health benefits (e.g. relief of undesirable gastrointestinal problems), the nutritionally acceptable composition will also provide energy like any other food substance. A nutritionally acceptable composition according to the invention is substantially different from compositions used in drug therapy, the nutritionally acceptable composition being composed of food ingredients (preferably natural ingredients) that are recognized as being safe, non-toxic to humans and substantially free of the side effects associated with typical prescription drugs (e.g. head ache, nausea, allergy, etc.).

Nevertheless, the nutritionally acceptable composition may further comprise additional safe and non-toxic components such as preservation agents, solubilizing agents, stabilizing agents, emulsifying agents, softening agents, coloring agents, odorous agents, antioxidant agents, etc.

The nutritionally acceptable composition can be presented as a solid form, as a dry form, or as a liquid form for oral administration. The nutritionally acceptable composition can be presented in a variety of ingestible forms of food or food supplements, including but not limited to milk, yogurt, curd, fermented milks, milk-based fermented products, soy based fermented products, fermented cereal based products, milk based powders, infant formulae, protein concentrates such as those used in hospitals, etc.

In embodiments the nutritionally acceptable composition comprises the combination of Lactobacilli and also fermented proteins including, but not limited to, fermented soy proteins, fermented milk proteins, fermented rice proteins, fermented pea proteins, fermented hemp proteins, fermented almond proteins and fermented insect proteins (e.g. larvae proteins).

The combination of Lactobacilli and the nutritionally acceptable composition may be integrated into any suitable support for oral delivery, for instance a gel, a capsule, a tablet, a suspension, or any other suitable support known to the person skilled in the art. Preferably the amount of Lactobacilli included in a single capsule, in a single tablet, in a certain volume of suspension or the like is in the range of about 10 billion to 200 billion.

The invention also encompasses kits and containers comprising multiple doses of the nutritionally acceptable composition, including for instance blister packages, reclosable bottles and the like comprising a certain amount of the composition (e.g. 25 ml, 50 ml, 100 ml or more) or a number of capsules or tablets (e.g. 10, 15, 25, 50 or more). Such kit or container can advantageously include instructions in the form of a pamphlet or of any other printed support, indicating the quantities of the composition to be administered, the instructions for the administration, the instructions to mix the components (e.g. if in powder form), etc.

The manufacture of a nutritionally acceptable composition according to the invention is within the skills of those in the art. For instance, the Lactobacilli may be incorporated into a suitable nutritionally acceptable vehicle. Alternatively a nutritionally acceptable composition comprising the combination of Lactobacilli can be obtained by fermenting live Lactobacilli bacteria in a suitable medium to obtain a ferment comprising the Lactobacilli and fermented proteins (e.g. fermented soy proteins, fermented milk proteins, fermented rice proteins, fermented peas proteins, fermented hemp proteins, etc.).

### EXAMPLE

The following example serves to illustrate the extent of the use of the present invention and not to limit its scope. Modifications and variations may be made without forgetting the intent and the extent of the invention. Even though other methods or equivalent products equivalent to those that are found herein to test or to realize the present invention may be used, the material and the preferred methods are described.

### Example 1: Lactobacillus acidophilus CL1285®, L. casei LBC80R® and L. rhamnosus CLR2® Improves QOL and Symptoms of IBS-C, IBS-D: Double-blind, Randomised, Placebo-controlled Study

The objectives of this clinical trial were to evaluate the effectiveness of a proprietary probiotic product, Lactobacillus acidophilus CL1285®+Lactobacillus casei LBC80R®+Lactobacillus rhamnosus CLR2® for relief of specific IBS-related symptoms, improvement in quality of life, effect on stool consistency and frequency, and attainment of adequate relief (AR) in otherwise healthy adults with irritable bowel syndrome of the constipation (IBS-C), diarrhea (IBS-D) and mixed (IBS-M) subtypes.

### MATERIALS AND METHODS

### Experimental Design, Study Implementation, and Data Collection

The protocol of this prospective, double-blind, randomised, placebo-controlled study was approved by an independent IRB, IntegReview. All participating subjects signed an informed consent. Subjects aged 18 years or older were recruited at 3 clinical study sites located in California, USA.

Subjects ingested 2 capsules of Active or Placebo product with breakfast each day. Each Active capsule contained 50 billion c.f.u. of live organisms (L.acidophilus CL1285®, L.casei LBC80®, and L.rhamnosus CLR2®) plus inert ingredients. The Placebo capsules contained the inert ingredients only.

Subjects were required to have met the Rome III criteria for IBS (Shih and Kwan, 2007). The Rome III criteria include presence of recurrent abdominal pain or discomfort at least 3 days/month in the last 3 months, associated with 2 or more of the following: improvement with defecation, onset associated with a change in frequency of stool, and onset associated with a change in form (appearance) of stool. Symptom onset must be at least 6 months prior to diagnosis.

Subjects were required to complete a 7-day placebo run-in period to demonstrate compliance with intake of investigational product (IP) and completion of daily diaries documenting IP consumption, stool frequency, stool consistency as defined by the Bristol Stool Chart (BSC), pain severity, and concomitant medications. Successful completion of the run-in period also required presence of abdominal pain on at least 2 days, associated with at least 2 of the following: improvement with defecation, onset associated with a change in frequency of stool, and onset associated with a change in the form or appearance of the stool. Potential subjects with diagnosed gastrointestinal disease other than IBS, prior abdominal surgery or systemic disease with the potential to confound study results or compromise safety, life expectancy less than 6 months, pregnancy or breastfeeding, lactose intolerance, immunodeficiency, eating disorder, recent use of antibiotics, allergy to the study product, or daily consumption of probiotics, fermented milk, or yogurt were excluded. Following successful completion of the run-in period, 113 subjects were randomised in a 2:1 ratio to Active study product or Placebo.

Subjects returned to the study site at 6-week intervals for a total of 12 study weeks. At each visit, subjects completed two questionnaires, the IBS-SSS (Symptom Severity Scale) and the IBS-QOL (Quality of Life, which includes an overall score and assessment of QOL in eight validated domains: Dysphoria, Interference with Activity, Body Image, Health Worry, Food Avoidance, Social Reaction, Sexual, and Relationship; see “Information Sheet on the Irritable Bowel Syndrome-Quality of Life Measure(IBS-QOL)” published by the University of Washington that is available on the web).

Subjects were questioned at each visit as to whether they had had adequate relief of their IBS symptoms. Subjects continued to record stool consistency and frequency, symptom severity, IP consumption, and concomitant medications in diaries, which were collected at each visit and reviewed for legibility and completeness. Returned IP was counted to evaluate compliance, and new IP was issued at Visit 3. Subjects were questioned about any adverse events (AEs) noted in the diary to determine onset and recovery dates and severity. Reported AEs were subsequently classified as to relationship to IP (related, possibly related, unlikely to be related, not related) by the investigator.

### Study Endpoints

Study endpoints included change in abdominal pain score, distention score, days with pain, score improvements on the IBS-SSS and IBS-QOL (including the QOL domains), and AR. Changes in stool frequency and stool consistency over the study period were examined within IBS subtypes and within subgroups of IBS subtype and gender. Safety endpoints were the incidence, severity, and relationship of IP to reported adverse events.

### Study Populations

A modified intent-to-treat (mITT) population was defined as subjects who were randomised and received at least one dose of IP; this population was used for the efficacy analysis and the safety analysis.

### Data Management

Data were collected on hard-copy source documents at the study sites and entered into a web-based relational database. On-site monitoring of 100% of clinical data fields against the source document was completed by clinical research associates (CRAs); queries were generated as needed for resolution by site clinical team. After all the data had been entered and all queries resolved, the database was hard-locked for analysis. Data files were then extracted by the study biostatistician and the subject ID numbers were matched with their treatment assignments to unblind the study.

### Statistical Analysis

The number of subjects screened, number randomised, number withdrawn early, and number completed were tabulated by treatment group.

The mITT population as a whole was analysed for symptom endpoints and QOL endpoints, along with subpopulations of IBS subtype and gender. Changes in stool consistency and frequency were analysed for the IBS-C and IBS-D subtypes and by gender within subtype.

Descriptive statistics were computed for baseline and demographic characteristics and tabulated by treatment group. Descriptive statistics included means, standard deviations, medians, ranges, and percentages, as dictated by the form of each variable. Inferential methods were not applied to baseline characteristics.

Compliance was calculated as percent of intended IP used, determined by returned bottle counts and subject diaries at Weeks 6 and 12, and compared across groups. Compliance was also defined as intake of 70% or more of intended IP, and analysed using the chi-square test.

Change in scores between Visit 2 and Visit 4 in the two treatment groups for the IBS-SSS, the IBS-QOL overall and domains, pain severity, days with pain in the last 10 days, distention severity, satisfaction with bowel habit, and interference of IBS with life in general were analysed. Stool consistency scores were assigned by subjects using the BSC and recorded in their subject diaries on a daily basis, and daily stool frequency was determined from the number of stools entered in the diary. Changes in median stool consistency and stool frequency during the 7-day run-in period vs. the last 7 days on study were compared. Stool consistency scores were expressed as median BSC scores per week, while stool frequency was expressed as median number of stools per day.

Data analysis revealed that the efficacy endpoints had to be evaluated within subtypes of IBS and for each gender separately, and many of the subgroup sample sizes were small. A large placebo effect was noted for many endpoints. We therefore elected to control the placebo effect by comparing change in the Active vs. Placebo groups; the mean improvement from Visit 2 to Visit 4 was calculated for each treatment group, and the Placebo value was then subtracted from the Active value, divided by the Placebo value, and multiplied by 100. For example, a mean change in pain severity of 15.0 in the Active group vs. a mean change of 10.0 in the Placebo group was reported as 50% improvement of Active over Placebo. This approach was used for comparing changes in the IBS-SSS, IBS-QOL and domains, pain severity, days with pain, distention severity, satisfaction with bowel habit, and interference of IBS with life in general. The same method was used to compare changes in stool consistency and frequency.

Analysis of change in stool consistency and frequency was carried out in subjects in the IBS-C and IBS-D subtypes and for male and female subjects within those subtypes. Within each subtype, “improvement” percentage was defined as the percentage change in the desirable direction for that subtype. Thus, the Results tables report “improvement” as a positive change for both subtypes, but the definition is different: for the IBS-C subtype an increase in mean BSC score (corresponding to softening of stools) and an increase in stool frequency were positive scores indicating improvement for that endpoint. For IBS-D subjects, a decrease in mean BSC score (indicating firmer stools) and a decrease in stool frequency were reported as improvement using positive numbers.

At the time the protocol was written, AR was a common primary endpoint in IBS trials, and was adopted as an endpoint for this trial. The endpoint IBS-AR had been shown to be a clinically and statistically relevant benefit in therapeutic IBS trials with alosetron (Camilleri et al. 1999), cilansetron, and tegaserod (Kellow et al., 2003; Tack et al., 2005). The AR consists of a single question: “Over the past week, have you had adequate relief of your IBS symptoms?”.

Safety was evaluated by calculating rates of subjects with adverse events in the Active and Placebo groups, and comparing them descriptively. Specific categories of adverse events were tabulated descriptively. Comparisons of subjects with specific adverse events were descriptive.

### RESULTS

A total of 113 subjects were enrolled, of which 86 subjects (76.1%) completed study. Completion rates were 73.0% in the Placebo group and 77.6% in the Active group.

Reasons for early discontinuation included loss to follow-up (10.6%), withdrawal of consent (7.1%), and other/unknown (6.1%). No subjects withdrew due to an adverse event.

### Demographics and Baseline Subject Characteristics

The distribution of demographic and baseline characteristics of the mITT population is presented in Table 1. The Placebo and Active groups were comparable in age, gender, and race.

TABLE 1
Demographics and Baseline Subject Characteristics at
Screening Visit, by Treatment Group, mITT Population

| | Placebo | | Active | |
|---|---|---|---|---|
| Age (years) | | | | |
| Mean | 39.9 | | 40.6 | |
| SD | 14.0 | | 13.4 | |
| n | 37 | | 76 | |
| Sex (#, %) | | | | |
| Male | 16 | (43.2%) | 29 | (38.2%) |
| Female | 21 | (56.8%) | 47 | (61.8%) |
| n | 37 | | 76 | |
| Race/ethnicity (#, %) | | | | |
| Caucasian, non-Hispanic | 14 | (37.8%) | 31 | (40.8%) |
| Asian | 2 | (5.4%) | 3 | (3.9%) |
| Hispanic | 7 | (18.9%) | 15 | (19.7%) |
| Native American | 1 | (2.7%) | 0 | |
| African-American or Black | 13 | (35.1%) | 25 | (32.9%) |

| | | | |
|---|---|---|---|
| Other | 0 | 2 | (2.6%) |

### Distribution of IBS Subtypes

The 113 patients were classified by the investigators at each site as IBS-C, IBS-D, or IBS-M based on their symptoms and history at study entry. The distribution of subjects in the three subtypes varied by clinical site, as shown in Table 2.

TABLE 2
Number and Percentage of Subjects in each IBS Subtype
by Investigational Site, mITT Population

| | IBS-C | IBS-D | IBS-M | Total |
|---|---|---|---|---|
| Garden Grove | 12 (75.0%) | 1 (6.3%) | 3 (18.6%) | 16 (100.0%) |
| San Francisco | 12 (23.5%) | 22 (43.1%) | 18 (34.6%) | 52 (100.0%) |
| Westlake | 16 (35.6%) | 29 (64.4%) | 0 | 45 (100.0%) |
| Total | 40 (35.7%) | 52 (46.4%) | 21 (18.6%) | 113 (100.0%) |

### Compliance

Subjects in the Placebo group consumed 87.0±17.8% of intended dose, while in the Active group consumption was 77.3±19.9%. Based on the protocol, consumption of at least 70% of intended IP, 84.4% of subjects in the Placebo group and 87.3% of subjects in the Active group were defined as compliant.

### IBS Symptom Severity Scale—IBS-SSS

The IBS-SSS consists of questions on severity of abdominal pain, number of days with pain in the last 10 days, severity of abdominal distention, satisfaction with bowel habit, and extent to which IBS interferes with the subject's life in general. All these except days of pain were scored on a Likert scale with a range of 0-100. When the overall score was computed, no mean improvement of 30% or more favoring the active groups was demonstrated.

In no subgroup of patients did the change in severity of abdominal pain reach 30% for the Active vs. the Placebo arm. However, clinical improvement was seen in many subgroups for the individual symptoms making up the IBS-SSS. Table 3 indicates that the highest percentage of improvement in the score of the IBS-SSS questions was seen in the IBS-D subtype, particularly in females, in whom improvement percentages varied from 50% to 144% in favour of the Active treatment. Males in the diarrhea subtype showed a smaller improvement in “satisfaction with bowel habit” (43%), and “interference with activity” (39%). Advantage in the IBS-C subtype was shown in “days with pain” in females (42%), and in “satisfaction with bowel habit” in both males and females (30% and 33%).

TABLE 3
Summary of Individual IBS-SSS Questions that
Showed Mean Differences of 30% or More
in Favour of Active Treatment,
mITT Population

| | | Improvement | | % |
|---|---|---|---|---|
| | | in Score, | | Improve- |
| | | V2 to V4 | | ment |
| Endpoint | | Mean ± SD (n) | | Active vs. |
| Symptoms | Group | Placebo | Active | Placebo |
| Days/ | IBS-M ♀♂ | 1.00 ± 1.00 | 2.25 ± 4.17 | 125% |
| pain | | (3) | (8) | |
| | IBS-C ♀ | 2.14 ± 2.97 | 3.03 ± 3.74 | 42% |
| | | (7) | (14) | |
| Distention | IBS-D ♀♂ | 11.31 ± 21.27 | 21.33 ± 23.82 | 89% |
| severity | | (13) | (27) | |
| | IBS-D ♀ | 16.56 ± 15.54 | 24.83 ± 24.65 | 50% |
| | | (9) | (18) | |
| Satisfaction | IBS-C ♀♂ | 23.27 ± 20.37 | 30.71 ± 24.12 | 32% |
| with | | (11) | (21) | |
| bowel | IBS-C ♀ | 24.71 ± 25.34 | 32.21 ± 24.20 | 30% |
| habit | | (7) | (14) | |
| | IBS-C ♂ | 20.75 ± 9.25 | 27.71 ± 25.57 | 33% |
| | | (4) | (7) | |
| | IBS-D ♀♂ | 23.00 ± 19.77 | 37.82 ± 30.95 | 64% |
| | | (13) | (27) | |
| | IBS-D ♀ | 21.44 ± 29.50 | 37.72 ± 35.81 | 76% |
| | | (9) | (18) | |
| | IBS-D ♂ | 26.50 ± 31.10 | 38.00 ± 19.58 | 43% |
| | | (4) | (9) | |
| | All | 24.61 ± 25.86 | 35.92 ± 29.66 | 46% |
| | Females | (18) | (40) | |
| Interfering | IBS-D ♀♂ | 16.00 ± 21.67 | 32.81 ± 26.91 | 105% |
| with | | (11) | (26) | |
| life | IBS-D ♀ | 14.38 ± 20.89 | 35.18 ± 30.58 | 144% |
| | | (8) | (17) | |
| | IBS-D ♂ | 20.33 ± 27.97 | 28.33 ± 18.91 | 39% |
| | | (3) | (9) | |
| | IBS-M ♀♂ | 13.00 ± 33.20 | 22.80 ± 23.64 | 75% |
| | | (4) | (10) | |
| | All | 16.88 ± 25.85 | 26.49 ± 30.73 | 57% |
| | Females | (17) | (40) | |

In many of the subgroups the percentage by which Active treatment outperformed Placebo on individual questions was considerably above our defined threshold of 30%.

### Quality of Life—IBS QOL Overall Scores

Overall scores on the IBS-QOL were examined for the total population, within IBS-C and IBS-D subtypes, and genders, and by gender within subtype. The percentage improvement in the Active vs. Placebo groups (Table 4) was comparable to the results obtained in IBS-SSS, with positive responses concentrated in the IBS-D subtype and in females. In males of the IBS-D subtype a lower degree of improvement (38%) for the Active was seen.

TABLE 4
Summary of Improvement in Overall QOL
Score that Showed Mean Differences of 30%
or More in Favour of Active Treatment,
mITT

| | | Improvement in Score, | | % |
|---|---|---|---|---|
| | | Visit 2 to | | Improve- |
| | | Visit 4, | | ment, |
| Population | Subgroup | Mean ± SD (n) | | Active vs. |
| Group (n) | (n) | Placebo | Active | Placebo |
| Females | All (85) | 18.44 ± 23.15 (27) | 24.01 ± 22.39 (58) | 30% |
| and | IBS-D (37) | 15.44 ± 13.10 (11) | 25.40 ± 23.47 (26) | 65% |
| males | | | | |
| Females | All (55) | 11.03 ± 18.86 (17) | 21.40 ± 20.72 (38) | 94% |
| | IBS-C (20) | 10.29 ± 27.84 (7) | 20.70 ± 18.72 (13) | 101% |
| | IBS-D (25) | 12.78 ± 11.61 (8) | 22.40 ± 24.78 (17) | 75% |
| Males | IBS-D (12) | 22.55 ± 16.70 (3) | 31.05 ± 20.94 (9) | 38% |

### Quality of Life IBS-QOL Domain Scores

A therapeutic effect of Active IP over Placebo was demonstrated in female subjects for overall QOL scores (Table 4) and in each of the eight domains (Table 5). The effect in the female subgroup was observed in both the IBS-C and IBS-D subtypes. In the male IBS-D subgroup a therapeutic effect was seen for overall QOL score and in four domains.

TABLE 5
Summary for the eight IBS-QOL Domain Scores, mITT Population. Changes in Mean
Domain Scores that were 30% or Greater in Favor of Active Treatment are Underlined

| | Subtypes and Genders | | | | | | | | | |
|---|---|---|---|---|---|---|---|---|---|---|
| | All | | | | | | IBS-C | IBS-C | IBS-D | IBS-D |
| Domain | subjects | IBS-C | IBS-D | IBS-M | Female | Male | female | male | female | male |
| Dysphoria | 33.6 | 32.2 | 48.0 | 3.1 | 62.8 | 6.2 | 139.9 | −10.8* | 39.5 | 66.7 |
| lnterference | 42.9 | 27.8 | 78.1 | 8.9 | 208.4 | −9.4* | 1704.9 | −28.4* | 84.2 | 59.4 |
| with | | | | | | | | | | |
| activity | | | | | | | | | | |
| Body | 18.5 | −17.7* | 95.5 | −8.6* | 67.0 | −9.4* | 18.4 | −22.2* | 166.7 | 27.8 |
| image | | | | | | | | | | |
| Health | 45.2 | 19.1 | 112.2 | 46.6 | 80.3 | 19.1 | 32.8 | 11.1 | 131.8 | 19.1 |
| worry | | | | | | | | | | |
| Food | 3.5 | 29.9* | 73.5 | 37.2 | 82.5 | −33.0* | −25.2* | −33.7* | 641.9 | −31.4* |
| avoidance | | | | | | | | | | |
| Social | 36.4 | 65.3 | 29.5 | −15.1* | 112.6 | −7.3* | 2331.5 | −9.3* | 25.0 | 33.6 |
| reaction | | | | | | | | | | |
| Sexual | 35.7 | 35.0 | 13.4 | 583.9 | 88.8 | 1.9 | 59.3 | −9.3* | 16.5 | 0 |
| Relationship | 29.1 | 2.5 | 68.4 | 51.0 | 69.9 | 4.2 | 91.6 | −21.8 | 41.7 | 133.1 |

*A negative number indicates that improvement was greater in the Placebo group than in the Active group

### Adequate Relief

For the study population as a whole there was no difference between the two study groups with respect to AR of IBS symptoms at Visits 2, 3, and 4. A strong placebo effect was noted.

We additionally analysed data from each of the IBS subtypes to discover whether there were any differences in AR of IBS within the subtypes at any study visit. No differences were found between the two study groups in any of the three IBS subtypes. Analysis of subgroups of males and females, and subgroups by gender within each of the 3 IBS subtypes, yielded similar results.

### Stool Consistency

In the analysis of stool consistency, a positive change (“improvement”) indicates increased BSC score in IBS-C and decreased BSC score in IBS-D. Table 6 shows percent change, Active vs. Placebo, for subgroups with Active changes of 30% or more over Placebo.

TABLE 6
Subgroups That Showed Mean Differences of 30% or More for
Improvement in Stool Consistency, mITT Population

| | IBS | | | % I-mprove |
|---|---|---|---|---|
| | Subtypes | Treatment effect, | | ment, |
| | and | Visit 2 to Visit 4 | | Active vs. |
| Endpoint | Genders | Placebo (n) | Active (n) | Placebo |
| Stool | IBS-C | 1.06 ± 1.01 (4) | 1.81 ± 1.16 (8) | 71% |
| consistency | IBS-D | 0.93 ± 1.37 (7) | 1.78 ± 1.42 (20) | 91% |
| BSC | IBS-D | 0.95 ± 1.68 (5) | 1.88 ± 1.58 (13) | 98% |
| | IBS-D | 0.88 ± 0.18 (2) | 1.57 ± 1.13 (7) | 78% |

Median stool consistency improved for both the Placebo and Active treatment groups. Median changes in the Placebo group were typically about one BSC scale point, with a range from 0.88 to 1.50, and about 1.75 BSC scale points in the Active group, with a range from 1.17 to 1.88. Percent changes echoed those seen in endpoints presented earlier: males and females in the Active IBS-D subtype gave the largest response compared to Placebo. For males in the IBS-C subtype there was an advantage of Active over Placebo, but this was not seen for the IBS-C group overall, nor for females with IBS-C. The largest differences between the treatment groups were seen in the IBS-D subtype, in both males and females. The male subgroup and the subgroup of males with IBS-C also showed improvement in stool consistency vs. Placebo.

### Stool Frequency

In the analysis of stool frequency, a positive change (“improvement”) indicates increased frequency in IBS-C and decreased frequency in IBS-D. In both the Placebo and Active groups, stool frequency improved in both the IBS-C and IBS-D subtypes, with subjects in the IBS-C subtype having more frequent stools during their last week on study than during the run-in period, while subjects in the IBS-D subtype reported a decrease in stool frequency over that period. Table 7 shows the IBS subtypes and subgroups in which Active outperformed Placebo for stool frequency improvement by 30% or more.

TABLE 7
Subgroups that Showed Mean Differences of 30% or More
for Improvement in Stool Frequency, mITT Population

| Endpoint | | Improvement in Score | | % Improve- |
|---|---|---|---|---|
| Stool | | Visit 2 to Visit 4 | | ment, |
| frequency/ | | (mean, SD) | | Active vs. |
| day | Subtype | Placebo (n) | Active (n) | Placebo |
| | IBS-C | 0.27 ± 0.65 (11) | 0.77 ± 0.81 (22) | 185% |
| | IBS-C | 0.29 ± 0.49 (7) | 0.57 ± 0.76 (14) | 97% |
| | IBS-C | 0.25 ± 0.96 (4) | 1.13 ± 0.83 (8) | 352% |
| | IBS-D | 0.57 ± 2.28 (7) | 1.45 ± 1.76 (20) | 154% |
| | IBS-D | 1.20± (5) | 1.62 ± 2.02 (13) | 35% |
| | IBS-D | −1.00± no SD (2) | 1.14 ± 1.21 (7) | 214% |

### Site-Specific Effects

The Garden Grove clinical site had a particularly interesting subgroup of subjects: among the 16 subjects treated at Garden Grove, 12 were females with severe chronic constipation refractory to treatment. In this subgroup, mean daily stool frequency (an important endpoint for IBS-C, per the FDA guidance document) increased on the average 0.25 stools/day in the Placebo group and 0.75 stools/day in the Active subgroup, a 200% percentage increase for Active vs. Placebo. It is also noteworthy that for this clinical site the subjects randomised to Active treatment had fewer mean stools per week at baseline than subjects in the Placebo group (0.38 stools/day vs. 0.75 stools/day), making the greater stool increase in the Active group.

TABLE 8
Summary of Improvement in Daily Stool Frequency by Visit and Treatment
Group, Garden Grove Site with IBS Subtype Constipation, mITT Population

| | | | | | Improvement | | % |
|---|---|---|---|---|---|---|---|
| | | | | | (Visit 2 to | | Improvement, |
| | Visit 2 | | Visit 4 | | Visit 4) | | Active vs. |
| Stools/Day | Placebo | Active | Placebo | Active | Placebo | Active | Placebo |
| Mean | 0.75 | 0.38 | 1 | 1.13 | 0.25 | 0.75 | 200% |
| SD | 0.5 | 0.74 | 0 | 0.64 | 0.5 | 0.89 | |
| n | 4 | 8 | 4 | 8 | 4 | 8 | |

### Safety

A total of 7 subjects reported one or more AEs while on study; 3 of these subjects were in the Placebo group and 4 were in the Active group. A total of 14 AEs were reported by the 7 subjects; all were mild or moderate in severity except for severe cramping, reported by one subject in the Active group. Four events were judged by the Investigator to be probably related to study product: dry mouth with increased thirst, increased respiration, nausea, and fatigue. These events were all reported by one subject in the Placebo group; the dry mouth and increased thirst persisted throughout the study period but resolved the day before the subject's last study visit. No AEs were judged to be definitely related to study product, and there were no serious AEs.

### DISCUSSION

Discovery of an effective treatment for IBS has been the goal of drug and probiotic studies in recent years. While the subject populations of many probiotic studies have been small, several meta-analyses have been published, and certain probiotic species and strains have been shown to be more effective than others in mitigation of IBS symptoms (McFarland and Dublin, 2008; Ortiz-Lucas et al., 2013). The study of IBS is complicated by the fact that patients frequently demonstrate a psychological profile of anxiety and depression (Ford et al., 2014a); these psychological effects may be exacerbated by the absence of effective treatment and public perception of IBS as a non-serious condition. The effects of race, ethnicity, diet and culture must also be considered in the assessment of treatment (Fava et al., 2013; Hughes, 2012).

In this study the effect of combined L. acidophilus CL1285®, L.casei LBC80R® and L. rhamnosus CLR2® was evaluated on symptoms of IBS and quality of life in 3 IBS subtypes: IBS-C, IBS-D, and IBS-M. As has been reported in many studies, the Placebo product used in this study produced improvements of IBS symptoms, reaching the level of a therapeutic effect such as described in the FDA guidance. In line with this guidance, an improvement of 30% of the Active product over Placebo was defined as a significant increased therapeutic value for the study endpoints change in QOL (overall and domains) and changes in abdominal pain, abdominal distention, days with pain, satisfaction with bowel habit, and interference with life in general. The strains in the Active product were effective vs. Placebo in simultaneously relieving clinical symptoms of IBS-C and IBS-D to varying degrees in both males and females. Stool frequency was improved in both subtypes; stool consistency, as measured by the BSC, improved in male and female subjects with IBS-D and in male subjects in the IBS-C subtype. These endpoints are currently those recommended by regulatory agencies in the United States and Europe to demonstrate efficacy in drug trials involving patients with IBS-C and IBS-D. These results show that these symptomatic benefits mirrored parallel trends in the IBS-QOL measure developed specifically for IBS (Drossman et al., 2000).

Female subjects, particularly of the IBS-D subtype, had a good response to the Active product in terms of stool frequency and consistency, and were the most responsive in terms of improvement in symptoms and QOL. While male response was also good in terms of stool frequency and consistency, the response to Active product over Placebo was less striking than in the female subgroup.

The symptomatic response observed in both IBS-C and IBS-D subtypes suggests that our 3 lactobacillus strains are effective in relieving symptoms and improving QOL in this indication.

The safety profile of the product used in this study has been documented in previous clinical trials (Beausoleil et al., 2007; Gao et al., 2010; Sampalis et al., 2010) and a quality improvement study (Maziade et al., 2015). The mechanism of action of the study product has been demonstrated in some intestinal pathology, but was not investigated in the present study (Auclair et al., 2015). Interestingly, the therapeutic gains observed with our 3 lactobacillus strains over placebo surpass those seen in drug studies, which are not free of significant adverse events (Cremonini et al., 2003; Kellow et al., 2003; Tack et al., 2005). Approved drugs have shown worse safety profiles than probiotic regimens, which have demonstrated an advantageous safety profile.

It is of interest to note that few studies have evaluated the effects of probiotics on QOL, and of those that did, many did not find a significant improvement (Halpern et al., 1996; Kellow et al., 2003; Kim et al., 2003; Moayyedi et al., 2010; Niv et al., 2005). A few studies showed improvement in some domains (Guglielmetti et al., 2011; Kajander et al., 2008; Lorenzo-Zúñiga et al., 2014; O'Mahony et al., 2005), but to our knowledge no effect on the “interference with activity” domain has been previously documented. O'Mahony et al. found lower IBS-QOL scores for L.salivarius ssp. and B.infantis for most domains (O'Mahony et al., 2005).

This study provides evidence of therapeutic effects in specific IBS subtypes and subgroups which were seen consistently for different endpoints: stool frequency and consistency, quality of life, improvement in distention severity, days with pain, and satisfaction with bowel habit. Our findings are in agreement with other studies conducted with probiotics and medications (Somberg, 2012).

A low incidence of AEs has been observed in previous studies conducted with the study product. This protocol was based on previous clinical studies conducted in Canada and the United States, with the optimal dosage of the product. The study product has also been previously evaluated in adults for antibiotic-associated diarrhea and C.difficile prevention, demonstrating a large reduction in diarrhea risk during a C.difficile outbreak in China (Gao et al., 2010). In the last decade of clinical research involving the study product, there have been no serious adverse events (SAEs) related to the study product in any of the clinical trials (Beausoleil et al., 2007; Gao et al., 2010; Maziade et al., 2015; Sampalis et al., 2010).

### CONCLUSIONS

The particular combination of live bacteria used in this study produced results which varied between genders and subtypes. Nevertheless it had a clear positive impact on stool consistency and frequency, quality of life, and IBS symptoms in both genders, without severe adverse events. These findings present a promising therapeutic option for subjects suffering from IBS.

### REFERENCES

Aagaard, K., Petrosino, J., Keitel, W., Watson, M., Katancik, J., Garcia, N., Patel, S., Cutting, M., Madden, T., Hamilton, H., Harris, E., Gevers, D., Simone, G., McInnes, P. and Versalovic, J., 2013. The Human Microbiome Project strategy for comprehensive sampling of the human microbiome and why it matters. Federation of American Societies for Experimental Biology Journal 27: 1012-1022.
Auclair, J., Frappier, M. and Millette, M., 2015. Lactobacillus acidophilus CL1285, Lactobacillus casei LBC80R, and Lactobacillus rhamnosus CLR2 (Bio-K+): Characterization, Manufacture, Mechanisms of Action, and Quality Control of a Specific Probiotic Combination for Primary Prevention of Clostridium difficile Infection. Clinical Infectious Diseases 60 Supplement 2: S135-143.
Beausoleil, M., Fortier, N., Guénette, S., L'ecuyer, A., Savoie, M., Franco, M., Lachaine, J. and Weiss, K., 2007. Effect of a fermented milk combining Lactobacillus acidophilus Cl1285 and Lactobacillus casei in the prevention of antibiotic-associated diarrhea: a randomized, double-blind, placebo-controlled trial. Canadian Journal of Gastroenterol 21: 732-736.
Camilleri, M., Mayer, E. A., Drossman, D. A., Heath, A., Dukes, G. E., McSorley, D., Kong, S., Mangel, A. W. and Northcutt, A. R., 1999. Improvement in pain and bowel function in female irritable bowel patients with alosetron, a 5-HT3 receptor antagonist. Alimentary Pharmacology & Therapeutics 13: 1149-1159.
Coffin, B., Dapoigny, M., Cloarec, D., Comet, D. and Dyard, F., 2004. Relationship between severity of symptoms and quality of life in 858 patients with irritable bowel syndrome. Gastroentérologie Clinique et Biologique 28: 11-15.
Cremonini, F., Delgado-Aros, S. and Camilleri, M., 2003. Efficacy of alosetron in irritable bowel syndrome: a meta-analysis of randomized controlled trials. Neurogastroenterology & Motility 15: 79-86.
Drossman, D. A., Patrick, D. L., Whitehead, W. E., Toner, B. B., Diamant, N. E., Hu, Y., Jia, H. and Bangdiwala, S. I., 2000. Further validation of the IBS-QOL: a disease-specific quality-of-life questionnaire. American Journal of Gastroenteroloy 95: 999-1007.
Ducrotté, P., Sawant, P. and Jayanthi, V., 2012. Clinical trial: Lactobacillus plantarum 299v (DSM 9843) improves symptoms of irritable bowel syndrome. World Journal of Gastroenterology 18: 4012-4018.
Fava, F., Gitau, R., Griffin, B. A., Gibson, G. R., Tuohy, K. M. and Lovegrove, J. A., 2013. The type and quantity of dietary fat and carbohydrate alter faecal microbiome and short-chain fatty acid excretion in a metabolic syndrome ‘at-risk’ population. International Journal of Obesity (London) 37: 216-223.
Ford, A.C. and Moayyedi, P., 2010. Meta-analysis: factors affecting placebo response rate in the irritable bowel syndrome. Alimentary Pharmacology & Therapeutics 32: 144-158.
Ford, A. C., Quigley, E. M., Lacy, B. E., Lembo, A. J., Saito, Y. A., Schiller, L. R., Soffer, E. E., Spiegel, B. M. and Moayyedi, P., 2014a. Effect of antidepressants and psychological therapies, including hypnotherapy, in irritable bowel syndrome: systematic review and meta-analysis. American Journal of Gastroenterology 109: 1350-1365; quiz 1366.
Ford, A. C., Quigley, E. M., Lacy, B. E., Lembo, A. J., Saito, Y. A., Schiller, L. R., Soffer, E. E., Spiegel, B. M. and Moayyedi, P., 2014b. Efficacy of prebiotics, probiotics, and synbiotics in irritable bowel syndrome and chronic idiopathic constipation: systematic review and meta-analysis. American Journal of Gastroenterology 109: 1547-1561; quiz 1546, 1562.
Foxx-Orenstein, A., 2006. IBS—review and what's new. Medscape General Medicine 8: 20.
Gao, X. W., Mubasher, M., Fang, C. Y., Reifer, C. and Miller, L. E., 2010. Dose-response efficacy of a proprietary probiotic formula of Lactobacillus acidophilus CL1285 and Lactobacillus casei LBC80R for antibiotic-associated diarrhea and Clostridium difficile-associated diarrhea prophylaxis in adult patients. American Journal of Gastroenterology 105: 1636-1641.
Guglielmetti, S., Mora, D., Gschwender, M. and Popp, K., 2011. Randomised clinical trial: Bifidobacterium bifidum MIMBb75 significantly alleviates irritable bowel syndrome and improves quality of life—a double-blind, placebo-controlled study. Alimentary Pharmacology & Therapeutics 33: 1123-1132.
Halpern, G. M., Prindiville, T., Blankenburg, M., Hsia, T. and Gershwin, M. E., 1996. Treatment of irritable bowel syndrome with Lacteol Fort: a randomized, double-blind, cross-over trial. American Journal of Gastroenterology 91: 1579-1585.
Hoveyda, N., Heneghan, C., Mahtani, K. R., Perera, R., Roberts, N. and Glasziou, P., 2009. A systematic review and meta-analysis: probiotics in the treatment of irritable bowel syndrome. BMC Gastroenterology 9: 15.
Hughes, V., 2012. Microbiome: Cultural differences. Nature 492: S14-15.
Kajander, K., Myllyluoma, E., Rajilić-Stojanović, M., Kyronpalo, S., Rasmussen, M., Järvenpää, S., Zoetendal, E. G., de Vos, W. M., Vapaatalo, H. and Korpela, R., 2008. Clinical trial: multispecies probiotic supplementation alleviates the symptoms of irritable bowel syndrome and stabilizes intestinal microbiota. Alimentary Pharmacology & Therapeutics 27: 48-57.
Kellow, J., Lee, O. Y., Chang, F. Y., Thongsawat, S., Mazlam, M. Z., Yuen, H., Gwee, K. A., Bak, Y. T., Jones, J. and Wagner, A., 2003. An Asia-Pacific, double blind, placebo controlled, randomised study to evaluate the efficacy, safety, and tolerability of tegaserod in patients with irritable bowel syndrome. Gut 52: 671-676.
Kim, H. J., Camilleri, M., McKinzie, S., Lempke, M. B., Burton, D. D., Thomforde, G. M. and Zinsmeister, A. R., 2003. A randomized controlled trial of a probiotic, VSL#3, on gut transit and symptoms in diarrhoea-predominant irritable bowel syndrome. Alimentary Pharmacology & Therapeutics 17: 895-904.
Lorenzo-Zúñiga, V., Llop, E., Suarez, C., Alvarez, B., Abreu, L., Espadaler, J. and Serra, J., 2014.1.31, a new combination of probiotics, improves irritable bowel syndrome-related quality of life. World Journal of Gastroenterology 20: 8709-8716.
Lyra, A., Hillilä, M., Huttunen, T., Männikkö, S., Taalikka, M., Tennilä, J., Tarpila, A., Lahtinen, S., Ouwehand, A. C. and Veijola, L., 2016. Irritable bowel syndrome symptom severity improves equally with probiotic and placebo. World Journal of Gastroenterology 22: 10631-10642.
Maziade, P. J., Pereira, P. and Goldstein, E. J., 2015. A Decade of Experience in Primary Prevention of Clostridium difficile Infection at a Community Hospital Using the Probiotic Combination Lactobacillus acidophilus CL1285, Lactobacillus casei LBC80R, and Lactobacillus rhamnosus CLR2 (Bio-K+). Clinical Infectious Diseases 60 Supplement 2: S144-147.
McFarland, L. V. and Dublin, S., 2008. Meta-analysis of probiotics for the treatment of irritable bowel syndrome. World Journal of Gastroenterology 14: 2650-2661.
Moayyedi, P., Ford, A. C., Talley, N. J., Cremonini, F., Foxx-Orenstein, A. E., Brandt, L. J. and Quigley, E. M., 2010. The efficacy of probiotics in the treatment of irritable bowel syndrome: a systematic review. Gut 59: 325-332.
Niv, E., Naftali, T., Hallak, R. and Vaisman, N., 2005. The efficacy of Lactobacillus reuteri ATCC 55730 in the treatment of patients with irritable bowel syndrome—a double blind, placebo-controlled, randomized study. Clinical Nutrition 24: 925-931.
O'Mahony, L., McCarthy, J., Kelly, P., Hurley, G., Luo, F., Chen, K., O'Sullivan, G. C., Kiely, B., Collins, J. K., Shanahan, F. and Quigley, E. M., 2005. Lactobacillus and Bifidobacterium in irritable bowel syndrome: symptom responses and relationship to cytokine profiles. Gastroenterology 128: 541-551.
Ortiz-Lucas, M., Tobias, A., Saz, P. and Sebastian, J. J., 2013. Effect of probiotic species on irritable bowel syndrome symptoms: A bring up to date meta-analysis. Revista Espanola de Enfermedades Digestivas 105: 19-36.
Peery, A. F., Dellon, E. S., Lund, J., Crockett, S. D., McGowan, C. E., Bulsiewicz, W. J., Gangarosa, L. M., Thiny, M. T., Stizenberg, K., Morgan, D. R., Ringel, Y., Kim, H. P., Dibonaventura, M. D., Carroll, C. F., Allen, J. K., Cook, S. F., Sandler, R. S., Kappelman, M. D. and Shaheen, N. J., 2012. Burden of gastrointestinal disease in the United States: 2012 update. Gastroenterology 143: 1179-1187 e1171-1173.
Sampalis, J., Psaradellis, E. and Rampakakis, E., 2010. Efficacy of BIO K+ CL1285 in the reduction of antibiotic-associated diarrhea—a placebo controlled double-blind randomized, multi-center study. Archives of Medical Science 6: 56-64.
Shih, D. Q. and Kwan, L. Y., 2007. All Roads Lead to Rome: Update on Rome III Criteria and New Treatment Options. Gastroenterology Report 1: 56-65.
Somberg, J. C., 2012. The human microbiome and therapeutics. American Journal of Therapeutics 19: 247
Tack, J., Müller-Lissner, S., Bytzer, P., Corinaldesi, R., Chang, L., Viegas, A., Schnekenbuehl, S., Dunger-Baldauf, C. and Rueegg, P., 2005. A randomised controlled trial assessing the efficacy and safety of repeated tegaserod therapy in women with irritable bowel syndrome with constipation. Gut 54: 1707-1713.
Whelan, K. and Myers, C. E., 2010. Safety of probiotics in patients receiving nutritional support: a systematic review of case reports, randomized controlled trials, and nonrandomized trials. American Journal of Clinical Nutrition 91: 687-703.
Yoon, H., Park, Y. S., Lee, D. H., Seo, J. G., Shin, C. M. and Kim, N., 2015. Effect of administering a multi-species probiotic mixture on the changes in fecal microbiota and symptoms of irritable bowel syndrome: a randomized, double-blind, placebo-controlled trial. Journal of Clinical Biochemistry and Nutrition 57: 129-134.

### * * *

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a compound” includes one or more of such compound, and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. (canceled)

2. A method for improving quality of life of a subject suffering from irritable bowel syndrome (IBS), comprising administering to said subject a combination of live Lactobacillus acidophilus, live Lactobacillus casei, and live Lactobacillus rhamnosus, wherein said administration provides to said subject at least one benefit selected from the group consisting of: satisfaction with bowel habit, minimal interference of IBS with normal activity, improved body image, reduction of food avoidance, increased social reaction, reduced sexual dysfunction, and improved relationships.

3. The method of claim 2, wherein said Lactobacillus acidophilus is Lactobacillus acidophilus CL1285® deposited at the CNCM, wherein said Lactobacillus casei is Lactobacillus casei LBC80R® deposited at the CNCM, and wherein said Lactobacillus rhamnosus is Lactobacillus rhamnosus CLR2® deposited at the CNCM.

4. The method of claim 2, wherein said administering comprises administration of a nutritionally acceptable composition comprising said combination of Lactobacilli.

5. The method of claim 2, wherein said administering comprises administering at least 10 to 200 billion of said combination of Lactobacillus.

6. The method of claim 2, wherein said combination of live Lactobacillus acidophilus, live Lactobacillus casei, and live Lactobacillus rhamnosus comprises about 1-10% L. acidophilus, 70-90% L. casei and about 5-20% L. rhamnosus of the colony forming units (CFU) of the combination.

7. The method of claim 2, wherein said administering comprises administering said combination of Lactobacilli at least once a day.

8. The method of claim 2, wherein said administering comprises administering a capsule comprising said combination of Lactobacilli.

9. The method of claim 2, wherein said administering comprises administering a ferment, said ferment comprising fermented proteins in addition to said combination of Lactobacilli.

10. The method of claim 9, wherein said fermented proteins are selected from the group consisting of fermented soy proteins, fermented milk proteins, fermented rice proteins, fermented pea proteins, fermented hemp proteins, fermented almond proteins and fermented insect proteins.

11. A method for the relief of irritable bowel syndrome (IBS) in a human subject in need thereof, comprising administering to said subject a combination of live Lactobacillus acidophilus CL1285®, live Lactobacillus casei LBC80R® and live Lactobacillus rhamnosus CLR2®.

12. The method of claim 11, wherein said IBS comprises at least one of constipation-predominant IBS (IBS-C), diarrhea-predominant IBS (IBS-D), and mixed bowel patterns IBS (IBS-M).

13. The method of claim 11, wherein said method provides simultaneous relief of multiple symptoms of IBS.

14. The method of claim 13, wherein said symptoms are selected from the group consisting of abdominal pain, bloating and constipation.

15. The method of claim 11, wherein said administering comprises administration of a nutritionally acceptable composition comprising said combination of Lactobacilli.

16. The method of claim 11, wherein said administering comprises administering at least 10 to 200 billion of said combination of Lactobacillus.

17. The method of claim 11, wherein said combination of live Lactobacillus acidophilus, live Lactobacillus casei, and live Lactobacillus rhamnosus comprises about 1-10% L. acidophilus, 70-90% L. casei and about 5-20% L. rhamnosus of the colony forming units (CFU) of the combination.

18. The method of claim 11, wherein said administering comprises administering said combination of Lactobacilli at least once a day.

19. The method of claim 11, wherein said administering comprises administering a capsule comprising said combination of Lactobacilli.

20. The method of claim 11, wherein said administering comprises administering a ferment, said ferment comprising fermented proteins in addition to said combination of Lactobacilli.

21. The method of claim 20, wherein said fermented proteins are selected from the group consisting of fermented soy proteins, fermented milk proteins, fermented rice proteins, fermented pea proteins, fermented hemp proteins, fermented almond proteins and fermented insect proteins.

22.-45. (canceled)
